(19) United States
(12) Patent Application Publication
(10) Pub. No.: US 2009/0082619 A1
(43) Pub. Date: Mar. 26, 2009

(54) METHOD OF TREATING CARDIOMYOPATHY

(76) Inventor: Eduardo DE MARCHENA, Miami, FL (US)

Correspondence Address: NIXON & VANDERHYE, PC, 901 NORTH GLEBE ROAD, 11TH FLOOR, ARLINGTON, VA, 22203, US

(21) Appl. No.: 12/326,432

(22) Filed: Dec. 2, 2008

Related U.S. Application Data
(63) Continuation-in-part of application No. 11/920,365, filed on Nov. 14, 2007.
(60) Provisional application No. 60/688,730, filed on Jun. 9, 2005.

(51) Int. Cl. A61B 17/00

(52) U.S. Cl. 600/37

(57) ABSTRACT

The surgical implantation of a link, which may be in the form of a tether or a looped band, is proposed to connect and reduce the spacing between facing walls of the left ventricle, to reduce dilation of the left ventricle. Decreasing the distance between the facing portions of the ventricle may also more appropriately align the chordal apparatus to decrease mitral regurgitation. The implanted link thus improves heart function by reducing left ventricular failure.

### CROSS-REFERENCE TO RELATED APPLICATION

This application is a continuation-in-part of U.S. application Ser. No. 11/920,365, which was filed Nov. 14, 2007, which is a U.S. National Stage of PCT Application No. PCT/US2006/019496, which was filed on May 19, 2006, which designated the U.S., and which claims the benefit of the filing date of U.S. Provisional Application No. 60/688,730, which was filed on Jun. 9, 2005, the entire contents of each of which are hereby incorporated by reference.

### BACKGROUND OF THE TECHNOLOGY

Ischemic and Non Ischemic Dilated Cardiomyopathy causes the heart to become enlarged and to function poorly. Some people have stable disease and there is little worsening of their condition. Others have progressive disease. As a result, the muscle of the heart becomes weak, thin or floppy and is unable to pump blood efficiently around the body. This typically causes fluid to build up in the lungs which therefore become congested, resulting in a feeling of breathlessness. This is referred to as congestive (left) heart failure. Often there is also right heart failure which causes fluid to accumulate in the tissues and organs of the body, usually the legs and ankles, and the liver and abdomen. Left ventricular dilation can also lead to secondary Mitral valvular regurgitation, further worsening cardiac performance, referred to as functional Mitral Regurgitation.

The typical pathology of Dilated Cardiomyopathy includes dilation of the ventricle and contraction deficiency, and heart failure systems appear in 75 to 95% of patients, often with complications of arrhythmic-death (sudden death) or thrombosis and embolism during the course of the disease. It is an intractable disease with a mortality rate of approximately 50% within 5 years of onset. This disease also accounts for the majority of heart transplant patients in Europe and the United States.

### BRIEF SUMMARY OF THE TECHNOLOGY

The present disclosure proposes the surgical implantation of a link, which may be in the form of a tether or a looped band, to reduce a transventricular size and to improve the ventricular geometry to thereby mitigate the affects of dilated cardiomyopathy.

In some embodiments, a tether or looped band would connect opposing papillary muscles.

In alternate embodiments, a tether or looped band could connect one of the papillary muscles to the inter-ventricular septum.

In another alternate embodiment, a tether or looped band could connect one of the papillary muscles to an exterior wall of the left ventricle.

In yet another alternate embodiment, a tether or looped band could connect the inter-ventricular septum to an exterior wall of the left ventricle.

In each of the above discussed embodiments, the tether or looped band would act to reduce dilation and improve heart function by reducing left ventricular failure and decreasing mitral valvular regurgitation

A percutaneously delivered trans-vascular device is proposed to enable the surgeon to implant the tether or looped band in the left ventricle. The trans-vascular device may be inserted through the femoral vein and delivered to the left ventricle via a trans-septal approach into the left atrium, across the mitral valve and into the left ventricle. Alternatively, the device could be inserted into the femoral artery and then, through a retrograde course, be advanced through the aortic valve and to the left ventricle. The device will allow attachment of a tether to opposite sides of the left ventricle, to thereby draw together the respective walls of the left ventricular cavity. As an alternative to the trans-vascular approach, the tether can be attached during an open heart surgical procedure. In yet another alternate method, one could perform a trans-ventricular apical approach through a small incision in the chest wall.

A method of treating dilated cardiomyopathy could include: securing at least one tether structure to opposed, facing portions of a ventricle of the heart of a patient having dilated cardiomyopathy; and reducing a length of said at least one tether structure so as to draw said facing portions of the ventricle towards each other to reduce a transventricular dimension of said heart.

A method of reducing a transventricular size and geometry in a patient having dilated cardiomyopathy could include: securing at least one tether structure to opposed, facing portions of the left ventricle of said patient's heart; and reducing a distance between said facing portions by drawing said facing portions towards each other with said at least one tether structure to reduce a transventricular size and geometry of the patient's heart, thereby to mitigate the affects of the dilated cardiomyopathy. Decreasing the distance between the facing portions of the ventricle may also more appropriately align the chordal apparatus to decrease mitral regurgitation.

### BRIEF DESCRIPTION OF THE DRAWINGS

FIG. 1 is a schematic illustration of a normal four chamber heart;

FIG. 2 is a schematic illustration of a heart with a congenital false tendon;

FIG. 3 is a schematic illustration of a four chamber heart exhibiting Dilated Cardiomyopathy;

FIG. 4 is a schematic illustration of the four chamber heart of FIG. 3 wherein a link or band connects the papillary muscles so as to effect a reduction in the size of the left ventricular cavity;

FIG. 5 shows an example antegrade approach to the left atrium;

FIGS. 6-8 illustrate attachment of respective tethers or link portions to diametrically opposed papillary muscles of the left ventricle according to an example embodiment of the invention;

FIG. 9 illustrates the drawing together and attachment of the tethers or linked portions of FIG. 8 so as to draw the papillary muscles together to reduce the chamber of the left ventricle;

FIG. 10 illustrates the tethered or linked papillary muscles in an example embodiment of the invention;

FIG. 11 is a cross-sectional view illustrating a tether that connects a papillary muscle to the inter-ventricular septum;

FIG. 12 is a cross-sectional view illustrating a tether that connects an exterior wall of the left ventricle to the inter-ventricular septum; and

FIG. 13 is a cross-sectional view illustrating a tether that connects a papillary muscle to an exterior wall of the left ventricle.

### DETAILED DESCRIPTION OF THE INVENTION

As noted above, Dilated Cardiomyopathy is a condition wherein the heart has become enlarged and too weak to efficiently pump blood around the body causing a build up of fluid in the lungs and/or tissue.

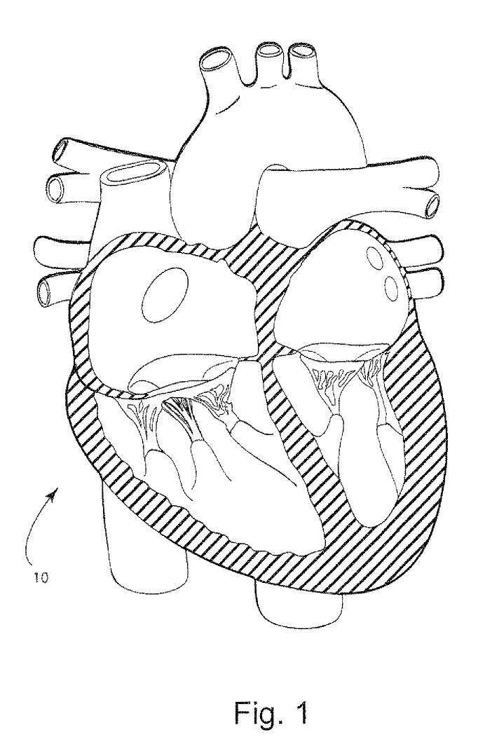

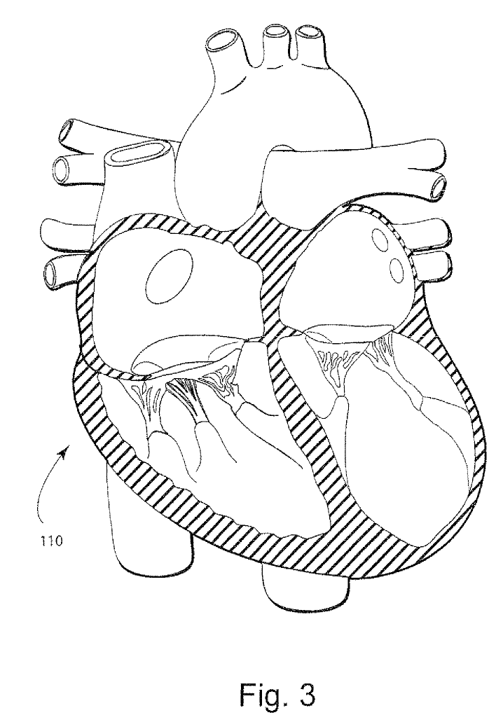

FIG. 1 illustrates a normal four chamber heart 10 whereas FIG. 3 illustrates the enlarged, thin walled heart 110 of a patient having Dilated Cardiomyopathy.

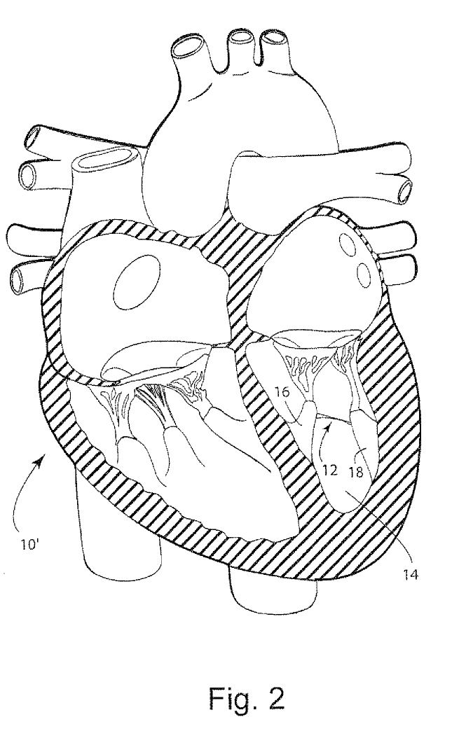

Referring to FIG. 2, some individuals have a congenital malformity of the heart in the form of a false tendon, more specifically, a left ventricular abnormal tendon 12 spanning the ventricular cavity 14 between the two papillary muscles 16, 18. or simply across opposing interior walls of the left ventricular cavity. This congenital malformation has no apparent affect on the function of an otherwise normal heart 10′. The inventor has observed, however, that patients with Dilated Cardiomyopathy that have this congenital false tendon appear to maintain a more favorable ventricular geometry, i.e., have less ventricular dilation, and consequently a more favorable clinical course than patients with Dilated Cardiomyopathy that lack this congenital false tendon.

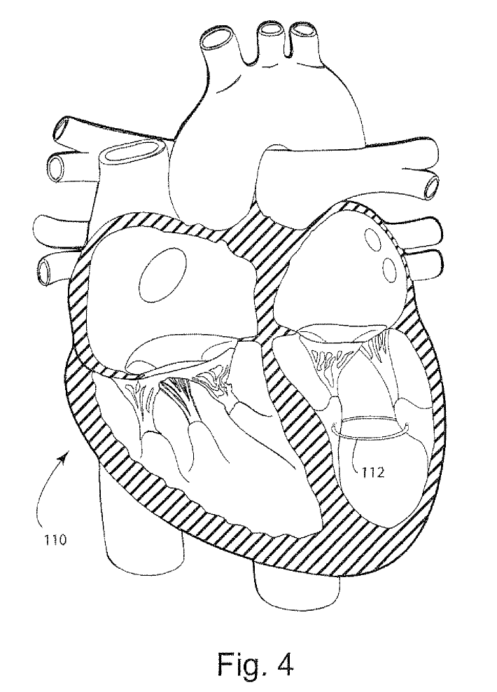

Consistent with this observation, the invention proposes the surgical or percutaneous interventional attachment of the two papillary muscles with a manufactured false tendon 112, as schematically illustrated in FIG. 4, to mimic the congenital false tendon structure 12, thereby to reduce dilation of the left ventricle 120 and consequently improve heart function, and improve clinical outcomes for patients with Dilated Cardiomyopathy.

Access to the left ventricle is preferably accomplished through the patient's vasculature in a percutaneous manner such that the vasculature is accessed through the skin remote from the heart, e.g., using a surgical cut down procedure or a minimally invasive procedure, such as needle access through use of the Seldinger technique, as is well known in the art. Depending upon the determined vascular access, the approach to the left ventricle may be antegrade, requiring entry into the left ventricle by crossing the interatrial septum and passing through the mitral valve. Alternatively, the approach can be retrograde where the left ventricle is entered through the aortic valve. As a further alternative an open surgical technique can be used with a trans-apical approach.

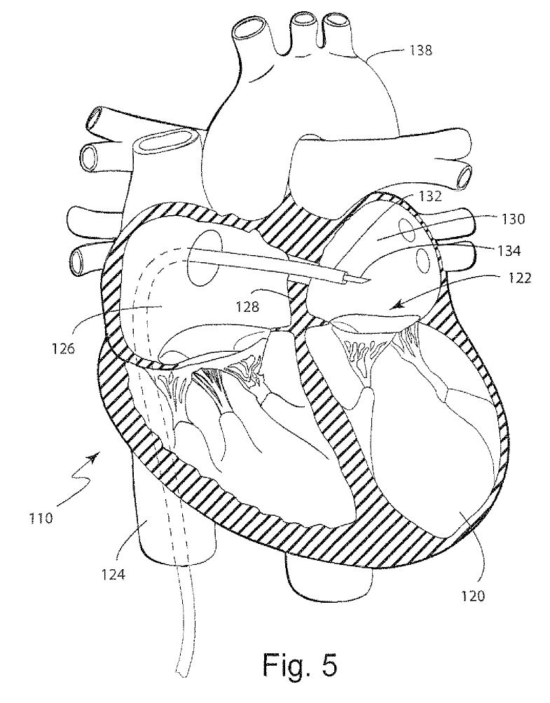

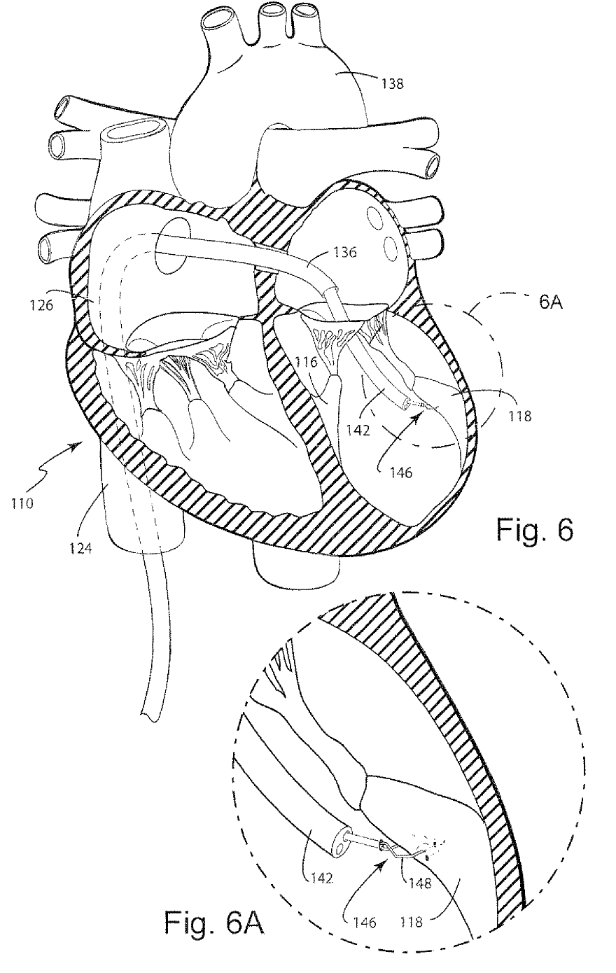

A typical antegrade approach to the left ventricle 120 through the mitral valve 122 is depicted in FIGS. 5-9. In this example embodiment, the left ventricle is accessed by inserting suitable elongated transvascular device(s) through the femoral vein, through the inferior vena cava 124, through the right atrium 126, across the interatrial septum 128, and into the left atrium 130. Thus, as shown in FIG. 5, a catheter 132 having a needle knife 134 may be advanced from the inferior vena cava 124 into the right atrium 126. Once the catheter 132 reaches the anterior side of the interatrial septum 128, the needle knife 134 is advanced so that it penetrates through the septum, e.g., at the fossa ovalis or the foramen ovale, into the left atrium 130. At that point, the catheter is advanced through the septum, a guide wire (not shown) is exchanged for the needle knife, and the catheter is withdrawn. As shown in FIG. 6, access through the interatrial septum 128 will usually be maintained by a placement of a guide catheter 136, e.g., over the guide wire which has been placed as described above. The guide catheter affords subsequent access to permit introduction of the instruments which will be used to engage and tether the papillary muscles, as described in more detail below.

As mentioned above, as an alternative to the presently preferred antegrade approach, a typical retrograde approach may be used. In such a case, the left ventricle 120 is accessed by an approach from the aortic arch 138, across the aortic valve (not shown), and into the left ventricle. The aortic arch may be accessed through a conventional femoral artery access route as well as through more direct approaches via the brachial artery, axillary artery or a radial or carotid artery. Again, such access may be achieved with the use of a guide wire over which a guide catheter may be fed to afford subsequent access to permit introduction of instruments as described in more detail below.

An advantage of the antegrade approach is that it eliminates any risks associated with crossing the aortic valve. Additionally, the antegrade approach permits the use of larger French catheter without the risk of arterial damage. On the other hand, the retrograde arterial approach eliminates the need for a trans-septal puncture, is an approach more commonly used by cardiologists, and provides direct access to the papillary muscles, without requiring that the mitral valve be crossed.

As will be appreciated, approaching the papillary muscles 116,118 for effective treatment requires proper orientation of the catheters, tools and the like throughout the procedure. Such orientation may be accomplished by steering of the catheter or tool to the desired location. In this regard, the guide catheter 136 may be pre-shaped to provide a desired orientation relative to the mitral valve, when the antegrade approach is used, or a desired orientation relative to the papillary muscles when the retrograde approach is used. For example, the guide catheter may have an L-shaped tip which is configured to direct instruments down into the left ventricle so that the tool or catheter is aligned with the axis of the mitral valve. Likewise the guide catheter may be configured so that it turns towards the papillary muscle(s) after it is placed over the aortic arch and through the aortic valve. In the alternative, the guide catheter, or the interventional instruments, may be actively steered, e.g., by having push/pull wires which permit selective deflection of the distal end in one of several directions, depending upon the number of pull wires, or by using other known techniques.

In an example embodiment of the invention, the papillary muscles 116,118 are grasped by partial or full penetration or piercing. This may be accomplished with a variety of grasping mechanisms, preferably including one or more piercing prongs extending from an instrument or catheter tool so as to grasp a target structure. Referring more specifically to the example embodiment of FIG. 6, an interventional tool 142 is fed through the guide catheter 136 to secure a first link portion or a tether structure 144 to one of the papillary muscles in the left ventricle. The deployment catheter or instrument is advanced from the distal end of the guide catheter 136 and may be observed in real time via any conventional imaging technique. In the illustrated example embodiment, a suture or clip applying instrument 142 is passed through the guide catheter 136. Advantageously, the instrument has a steerable tip so that it may be directed to a position in opposed facing relation to a target portion of a papillary muscle. Disposed at or adjacent the distal end of the instrument in this embodiment is a clamp or clip 146 for secure attachment to the respective papillary muscle. The clip or clamp is advanced out of the deployment catheter and into engagement with respective papillary muscle FIG. 6A schematically illustrates the distal end of the clip applicator instrument 142 with a loaded clip 146 of the tether structure 144 projecting there beyond, poised for application to the papillary muscle. The clip includes first and second arms 148 each terminating in a tissue penetrating or gripping tip 150 and a tether or suture 152 is secured to the proximal end of the clip 146. To secure the clip to the muscle, the distal end of one clip arm is contacted so as to engage the tissue. Then, the clip applicator 142 is manipulated so that the distal end of the other clip arm engages the tissue spaced from the first arm. The clip applicator is then actuated to close the clip 146 and clamp the tissue so as to secure the tether structure to the muscle, as shown in FIG. 7. Any suitable mechanism can be sued to close the clip. For example, a thin sheath could be advanced to close the clip into the papillary muscle and lock. If deemed necessary or desirable, one or more additional clips with tethers may be applied. The flexible tether(s) or suture(s) 152 extend proximally from the clip structure, as shown in FIG. 7, to be manipulated as described herein below to draw the papillary muscles together. In the illustrated embodiment, the tether or suture 152 is attached to the clip before deployment. However, the clip(s) may be applied first and the tether(s) attached thereafter to the clip(s).

Once the clip has been secured with respect to a first one of the papillary muscles, the instrument is withdrawn to reveal the flexible strand and the same or another instrument carrying another clip is conducted through the guide catheter adjacent the already placed flexible strand, as illustrated in FIG. 7. In the alternative, the instrument carries at least first and second clips and respective flexible strands so that the papillary muscles can be respectively engaged without withdrawing the instrument and reinserting it. Whether the clips are attached sequentially by the sequential feed of an instrument or sequentially by manipulating the instrument, after each papillary muscle has been engaged by respective clip(s) with respective flexible strand(s), the instrument is withdrawn through the guide catheter.

According to an alternate embodiment, non-absorbable suture loop(s) may be applied directly in the papillary muscles. For example, a variation of the Perclose A-T® vasculature closure device, which is a stitch knot transmitting device with a suture cutter could be used apply a suture loop. There are also known laparoscopic devices, such as the Quik-Stitch Endoscopic Suturing System, that may be adapted to transvascularly securing a tether to the papillary muscles.

As illustrated in FIG. 8, the guide catheter 136 remains in place with the flexible strands 152 extending therethrough from the respective secured clips 146. It is to be appreciated that if the retrograde approach is used instead, the strands would extend through a guide catheter disposed through the aortic valve, but the papillary muscles would otherwise be tethered in a like manner.

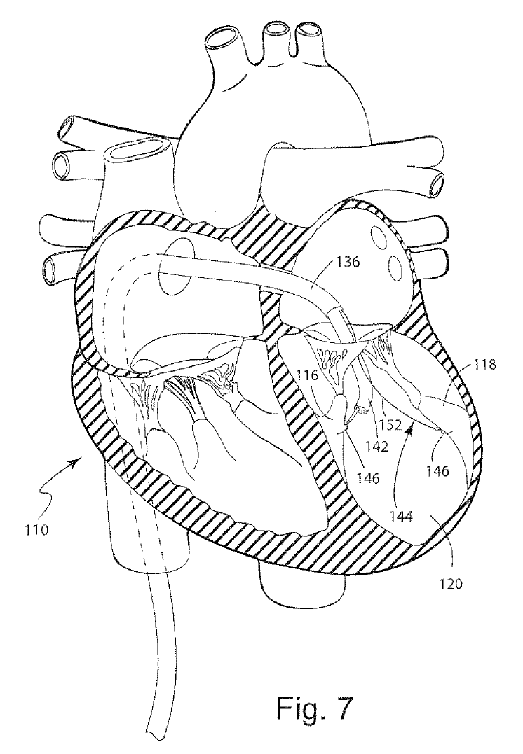

Referring now to FIG. 9, the tethered papillary muscles 116,118 are next drawn together by drawing the respective flexible tethers 152 together. In the illustrated example, an instrument 154 is advanced over the flexible tethers and the tethers are pulled through the instrument to draw the clips 146 toward one another. The tethers are then either tied or fastened together to define the desired spacing of the papillary muscles. For example, two tethers may have a knot transmitted to define the junction, or they are clipped to one another through the existing guiding catheter.

The tethering and drawing of the papillary muscles 116,118 towards one another may be conducted while monitoring the position of the muscles fluoroscopically, and under intra-cardiac ultrasound guidance, so that the papillary muscles 116,118 can be drawn to a desired transventricular distance. Intra cardiac Echo Doppler can also be used to assess the severity of mitral regurgitation, to adjust the length of the tethers to an optimum transventricular distance to suppress regurgitation. So apposing the papillary muscles reduces the size of the left ventricular cavity and will limit further distension of the ventricular wall, thereby mimicking the effect of the congenital false tendon to improve ventricular geometry and mitigate the effects of Dilated Cardiomyopathy.

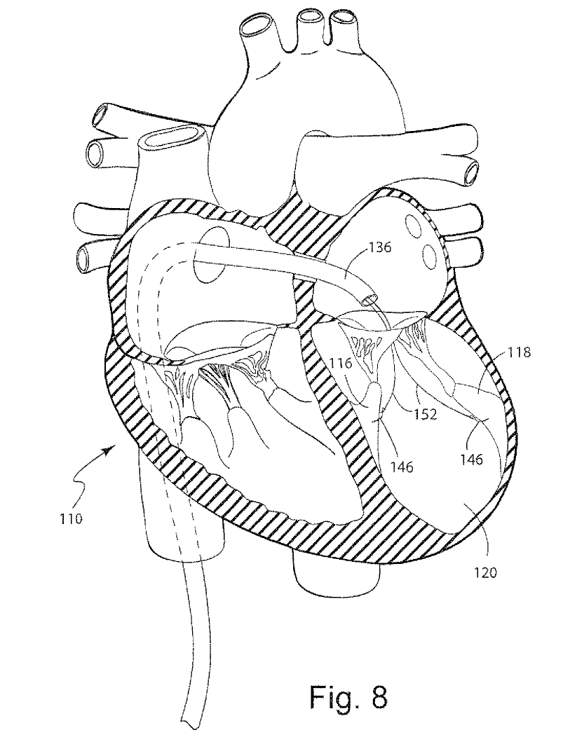

FIG. 10 illustrates the extra length flexible tether 152 removed. Any suitable instrument may be used to capture and sever the excess tether length such as, for example, a suture trimmer similar to that disclosed in US Published patent application number 20040097865, the disclosure of which is incorporated herein by this reference.

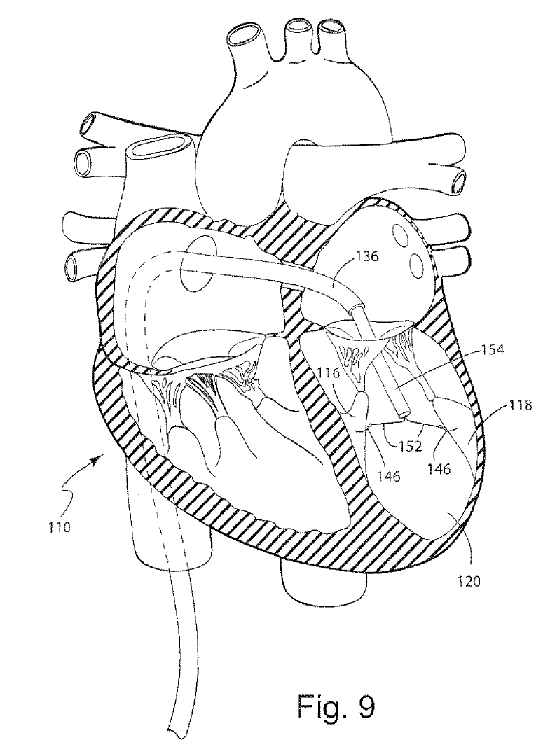

Alternate methods of attaching a tether to opposing structures within the left ventricle are illustrated in FIGS. 11-13. FIGS. 11-13 are cross-sectional views taken along the section line 11-11 in FIG. 10. The sectional views depict a cross-sectional view taken across the mid level of the right and left ventricles.

FIG. 11 shows a tether 162 which has been attached to one of the papillary muscles 118 and to the inter-ventricular septum 160.

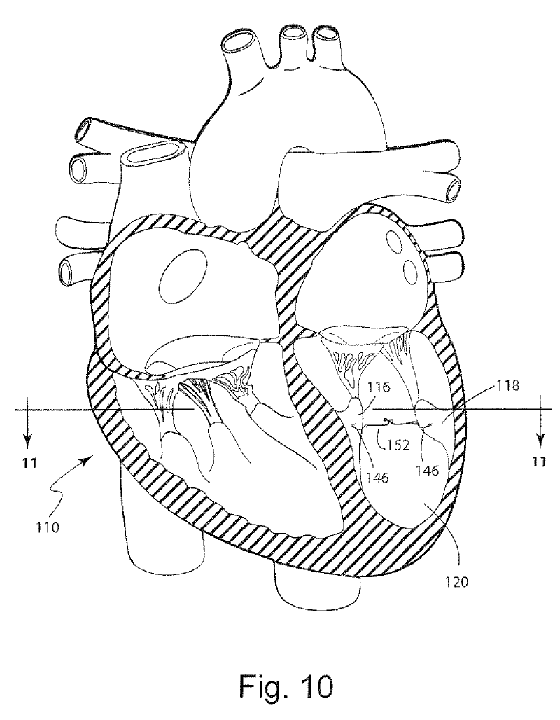

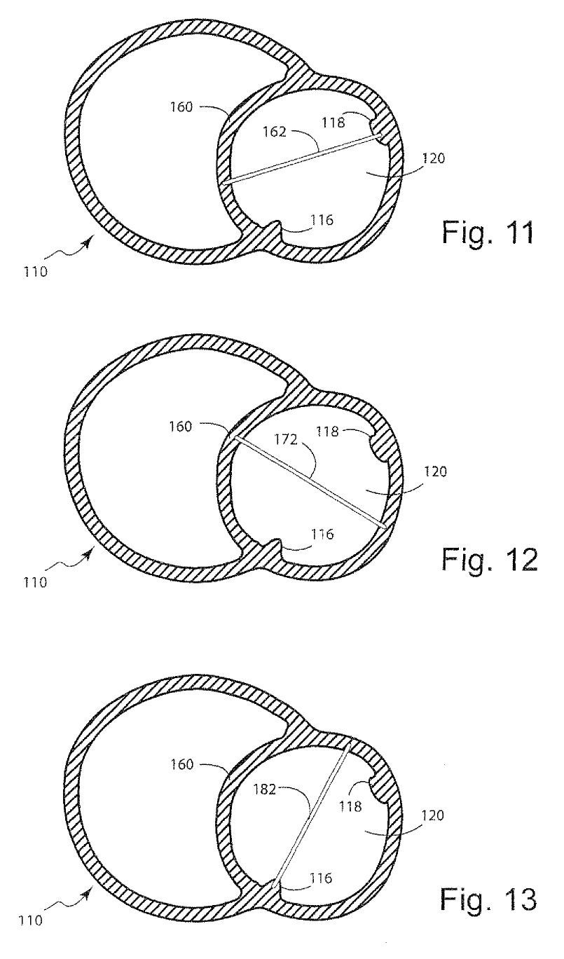

FIG. 12 shows another alternate embodiment in which a tether 172 is attached between the inter-ventricular septum 160 and an exterior wall of the left ventricle. FIG. 13 illustrates yet another alternate embodiment where a tether 182 is attached between another one of the papillary muscles 116 and the exterior wall of the left ventricle.

The tethers illustrated in FIGS. 11, 12 and 13 could also be inserted into the left ventricle using any of the methods described above. The placement of the tethers illustrated in FIGS. 11-13 can be used to draw facing portions of the ventricle towards each other to reduce a transventricular dimension of the heart, to thereby mitigate the affects of the dilated cardiomyopathy. Decreasing the distance between the facing portions of the ventricle may also more appropriately align the chordal apparatus to decrease mitral regurgitation. The tethers would limit further distention of the ventricular wall, thereby mimicking the effect of the congenital false tendon.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

What is claimed is:

1. A method of reducing a transventricular size and improving ventricular geometry in a patient having dilated cardiomyopathy, comprising:
securing a first end of a tether structure to one of the papillary muscles of the left ventricle of the patient's heart;
securing a second end of the tether structure to an opposing interior surface of the left ventricle of the patient's heart; and
reducing a distance between said papillary muscle and said opposing interior surface by drawing the papillary muscle and the opposing interior surface together with said at least one tether structure to reduce a transventricular size and geometry of the patient's heart, thereby to mitigate the affects of the dilated cardiomyopathy.

2. A method as in claim 1, wherein the opposing interior surface of the left ventricle is the inter-ventricular septum.

3. A method as in claim 2, wherein said tether structure comprises first and second suture filaments, wherein a clip is attached to an end of each suture filament, and wherein said securing steps comprise securing the clip of the first suture filament to the papillary muscle and securing the clip of the second suture filament to the inter-ventricular septum.

4. A method as in claim 3, further comprising advancing a clip applying device carrying the first and second suture filaments through said guide catheter.

5. A method as in claim 3, wherein the reducing step comprises:
drawing the clips of the first and second suture filaments together to thereby reduce a distance between said papillary muscle and said inter-ventricular septum; and
attaching ends of the suture filaments together.

6. A method as in claim 1, wherein the opposing interior surface of the left ventricle is an exterior wall of the left ventricle.

7. A method as in claim 1, wherein said tether structure comprises first and second suture filaments, wherein a clip is attached to an end of each suture filament, and wherein said securing steps comprise securing the clip of the first suture filament to the papillary muscle and securing the clip of the second suture filament to the exterior wall of the left ventricle.

8. A method as in claim 7, further comprising advancing a clip applying device carrying the first and second suture filaments through said guide catheter.

9. A method as in claim 7, wherein the reducing step comprises:
drawing the clips of the first and second suture filaments together to thereby reduce a distance between said papillary muscle and said exterior wall of the left ventricle; and
attaching ends of the suture filaments together.

10. A method as in claim 1, further comprising, before performing said securing steps, accessing the patient's vasculature remote from the heart, and advancing a guide catheter through the patient's vasculature so that a distal end thereof is disposed in the left ventricle of the patient's heart.

11. A method as in claim 10, further comprising, before advancing said guide catheter, creating a trans-septal opening with a needle knife disposed through a catheter.

12. A method as in claim 1, further comprising visualizing the papillary muscle and adjacent ventricular structures via fluoroscopy, or intra-cardiac ultrasound during said securing and reducing steps.

13. A method of reducing a transventricular size and improving ventricular geometry in a patient having dilated cardiomyopathy, comprising:
securing a first end of a tether structure to an exterior wall of the left ventricle of the patient's heart;
securing a second end of the tether structure to the inter-ventricular septum; and
reducing a distance between said exterior wall of the left ventricle and said inter-ventricular septum by drawing the exterior wall and the inter-ventricular septum together with said at least one tether structure to reduce a transventricular size and geometry of the patient's heart, thereby to mitigate the affects of the dilated cardiomyopathy.

14. A method as in claim 13, further comprising, before performing said securing steps, accessing the patient's vasculature remote from the heart, and advancing a guide catheter through the patient's vasculature so that a distal end thereof is disposed in the left ventricle of the patient's heart.

15. A method as in claim 14, further comprising, before advancing said guide catheter, creating a trans-septal opening with a needle knife disposed through a catheter.

16. A method as in claim 13, wherein said tether structure comprises first and second suture filaments, wherein a clip is attached to an end of each suture filament, and wherein said securing steps comprise securing the clip of the first suture filament to the exterior wall of the left ventricle and securing the clip of the second suture filament to the inter-ventricular septum.

17. A method as in claim 16, further comprising advancing a clip applying device carrying the first and second suture filaments through said guide catheter.

18. A method as in claim 16, wherein the reducing step comprises:
drawing the clips of the first and second suture filaments together to thereby reduce a distance between said exterior wall of the left ventricle and said inter-ventricular septum; and
attaching ends of the suture filaments together.

19. A method as in claim 13, further comprising visualizing the ventricular structures via fluoroscopy, or intra-cardiac ultrasound during said securing and reducing steps.
